(19) United States
(12) Patent Application Publication
(10) Pub. No.: US 2019/0309046 A1
(43) Pub. Date: Oct. 10, 2019

(54) SIGNAL TRANSDUCTION MODIFYING PROTEIN

(71) Applicant: AUTOLUS LIMITED, London (GB)

(72) Inventors: Shaun Cordoba, London (GB); Evangelia Kokalaki, London (GB); Vania Baldan, London (GB); Simon Thomas, London (GB); Maria Stavrou, London (GB)

(21) Appl. No.: 16/463,258

(22) PCT Filed: Nov. 27, 2017
(86) PCT No.: PCT/GB2017/053555

§ 371 Date: May 22, 2019

(30) Foreign Application Priority Data
Nov. 28, 2016 (GB) 1620070.1

(51) Int. Cl. C07K 14/73; C07K 14/705; C07K 16/28; C12N 9/12; C12N 9/16

(52) U.S. Cl. CPC C07K 14/70514; C07K 14/70517; C07K 16/2818; C12N 9/12; C12Y 207/11001; C07K 2319/02; C07K 2319/03; C07K 2319/33; C12Y 301/03048; C12N 9/16

(57) ABSTRACT

The present invention provides signal transduction modifying protein which comprises a domain which binds a phosphorylated immunoreceptor tyrosine-based inhibition motif (pITIM). The signal transduction modifying protein lacks a functional phosphatase domain. The present invention also provides cells which express such a signal transduction modifying protein, and cells which co-express such a signal transduction modifying protein together with a chimeric antigen receptor (CAR).

### FIELD OF THE INVENTION

The present invention relates to a signal transduction modifying protein (STMP) which binds a phosphorylated immunoreceptor tyrosine-based inhibition motif (pITIM). When expressed in a cell, the STMP competes with SHP-1 and/or SHP-2 for binding to pITIMs on inhibitory immune receptor molecules such as PD1. This reduces the de-phosphorylation of ITAM domains by SHP-1/SHP-2, thereby blocking or reducing the inhibition of immune activation mediated by these molecules.

### BACKGROUND TO THE INVENTION

Chimeric Antigen Receptors (CARs) graft the specificity of a monoclonal antibody (mAb) on to a T-cell. CAR T-cells targeted to CD19 have been tested in the clinical setting for the treatment of B cells malignancies and have shown promising results, particularly in B-cell acute lymphoblastic leukemia (B-ALL). Early studies of CD19 specific CAR therapy in B-ALL have shown response rates ranging from 70-91% at several institutions. However, the overall responses to CAR T-cell therapy in patients with other B cell malignancies such as CLL and lymphoma have been less impressive. Further, for the little clinical data that exists with CAR T-cells targeting solid cancer, response rates are even less. The reasons for these more modest responses are not entirely clear, but the tumour immune microenvironment is likely to play an important role.

The tumour immune microenvironment represents the background of immunological signals which a CAR T-cell encounters once it enters a tumour mass. This microenvironment is typically inhibitory and may consist of regulatory or suppressive cells, such as regulatory T-cells (Tregs) and myeloid derived suppressor cells (MDSCs), as well as inhibitory ligands that may bind to inhibitory receptors on T-cells and hinder T-cell anti-tumour responses, such as programmed death receptor (PD-1) and CTLA4.

Immune checkpoints refer to a multitude of inhibitory immune pathways which are important for maintaining self-tolerance and modulating the duration and amplitude of physiological immune responses in peripheral tissues in order to minimize collateral tissue damage. It is now clear that tumours co-opt certain immune-checkpoint pathways as a major mechanism of immune resistance, particularly against T cells that are specific for tumour antigens.

If CAR T-cell therapy is to be developed for solid tumours, strategies to overcome the immune checkpoint inhibition will be needed.

Various systems have been developed in an attempt to modulate immune checkpoint signals in the context of CAR T-cell therapy, as summarised in FIG. 2.

The simplest approach is the pre- or co-administration of blocking antibodies (FIG. 2A). Two main classes of antibodies have been used in this regard: those which block CTLA4, and those which block PD1/PDL1. The advantage of this approach is that it is technically simple and not only activates the CAR T-cells, but may also activate naturally occurring tumour-resident tumour specific T-cells. The disadvantage of this approach is the possibility of poor biodistribution of the antibodies into the tumour core and systemic toxicity since the antibody will effect T-cells everywhere. Also, immune-related adverse events (IRAEs) are frequent in patients treated with checkpoint blockade and have been reported to occur in up to 90% of patients treated with anti-CTLA-4 mAb and 70% of patients treated with anti-PD-1 mAb.

Other approaches which have been developed integrate the blockade into the CAR T-cells. For example, it is possible to engineer the CAR T cells such that they cause the localized secretion of checkpoint blocking scFvs (FIG. 2B). Alternatively, an immune checkpoint-relevant gene, such as the gene for the PD1 receptor, can be disrupted on CAR T-cells using genome editing tools, or silencing using siRNA (FIG. 2C). Alternatively, a “decoy” PD1 receptor can be expressed on the CAR T-cell. These cell integral approaches have the advantage that biodistribution is not limiting, and that there is no risk of systemic toxicity. The scFv secretion approach has the advantage that neighbouring native T-cells may also become activated.

However all of the approaches suggested to date suffer from the significant drawback that they block only one specific inhibitory receptor. In reality, there are a multitude of inhibitory pathways triggered by a multitude of different ligand:receptor interactions. The blocking of one inhibitory pathway makes it possible for the tumour to compensate for the specific immune checkpoint block using other molecules.

There is thus a need for an alternative approach to address the issue of checkpoint-mediated inhibition of CAR-T cells.

### DESCRIPTION OF THE FIGURES

FIG. 1 Diagram of immediate T-cell inhibition pathways. Activation of an inhibitory immune-receptor such as PD1 results in phosphorylation of ITIM domains. These are recognized by the SH2 domains of PTPN6. Upon recognition, PTPN6 is recruited to the juxta-membrane region and its phosphatase domain subsequently de-phosphorylates ITAM domains inhibiting immune activation.

FIG. 2—Schematic diagrams showing other approaches to avoiding inhibition of CAR-T cells by the tumour microenvironment and their relative advantages and disadvantages:

A. CAR T-cells administered with systemic PD1 or PDL1 blockade

Advantages: Simple, TILs also activated

Disadvantages: systemic toxicity, poor penetration of tumour core, single inhibitory receptor

B. CAR T-cells which are also engineered to secrete PD1 or PDL1 blocking scFv

Advantages: TILs also activated, better penetration of tumour core of blockade

Disadvantages: systemic toxicity still possible, single inhibitory receptor targeted

C. CAR T-cells which are also engineered to not express PD1

Advantages: No systemic toxicity

Disadvantages: TILs not activated. Only single inhibitory receptor targeted.

D. CAR T-cells which are also engineered to express a signal transduction modifying protein of the present invention

Advantages: No systemic toxicity. Multiple inhibitory receptors blocked

Disadvantages: TILs not activated.

FIG. 3—Diagram of a blocking signal system

A truncated PTPN6 which does not comprise a phosphatase domain is over-expressed, competing for full-length PTPN6 reducing ITIM signalling.

FIG. 4—Membrane localisation of the STMP can enhance the blocking effect because the STMP is effectively concentrated at the immunological synapse

a) a schematic diagram illustrating the inhibition of T-cell activation via PTPN6

b) PTPN6-mediated inhibition is blocked by a membrane-tethered STMP

FIG. 5—PD-1 signal blockade using truncated SHP-1 (PTPN6) or truncated SHP-2 PBMC cells were cotransduced with PD1 and either CAR alone (FMC63); or a bicistronic construct containing CAR and truncated SHP-1, or CAR and truncated SHP-2. These cells were co-cultured for 48 hours with SupT1 cells transduced with CD19, PDL1 or both and IFNγ release measured by ELISA.

FIG. 6—Schematic diagram illustrating the constructs tested in Example 2

### SUMMARY OF ASPECTS OF THE INVENTION

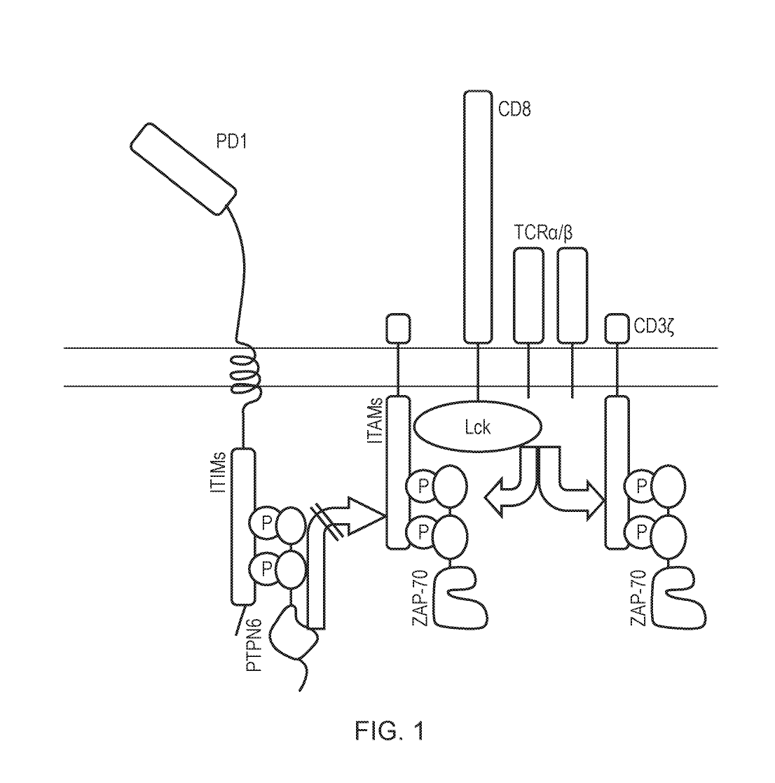

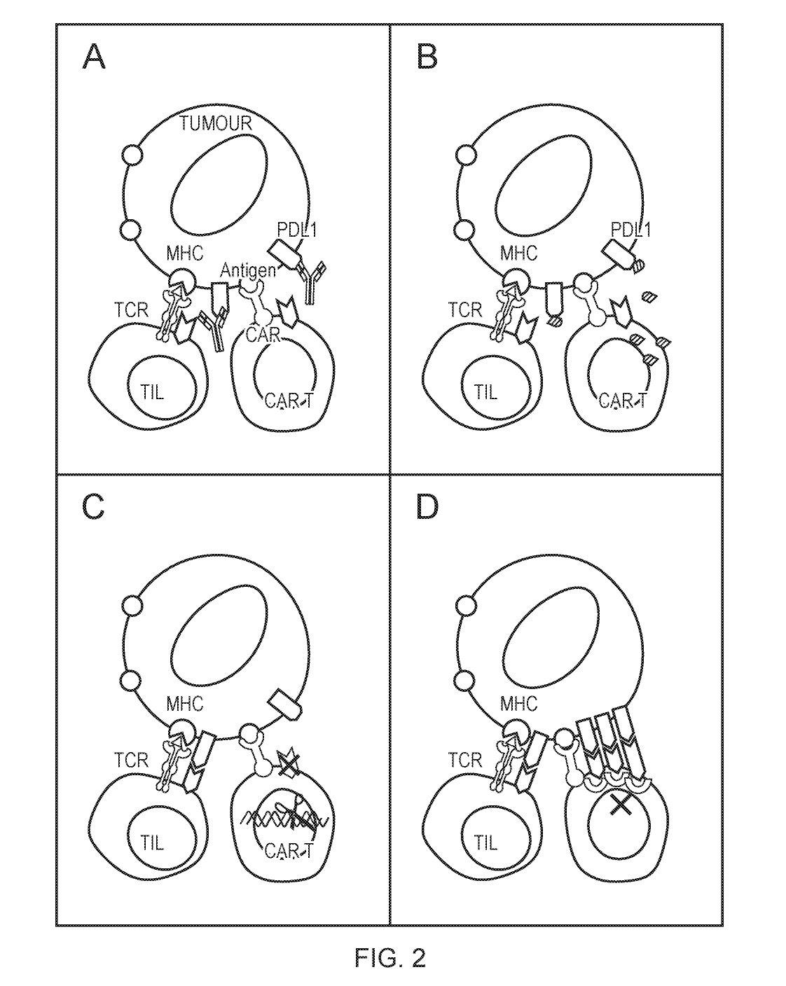

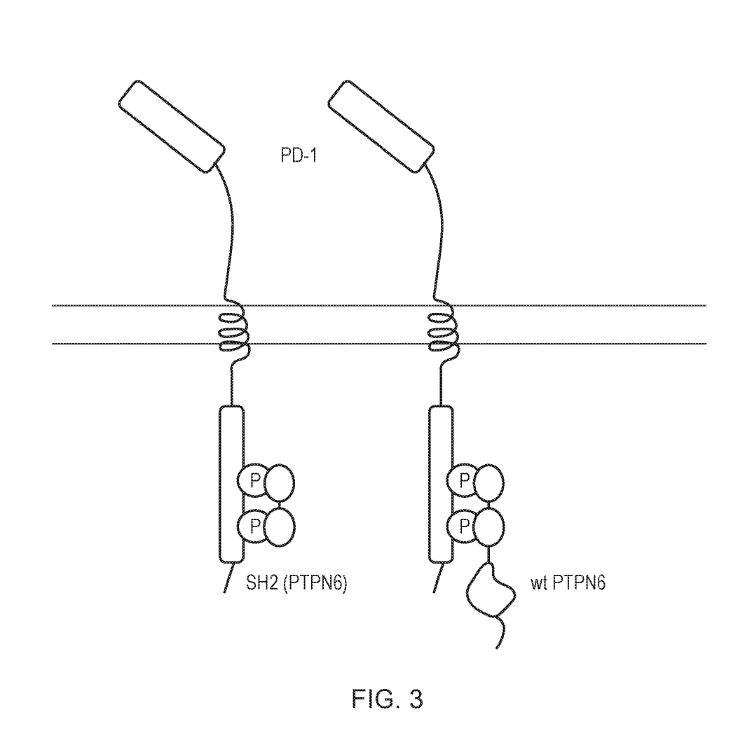

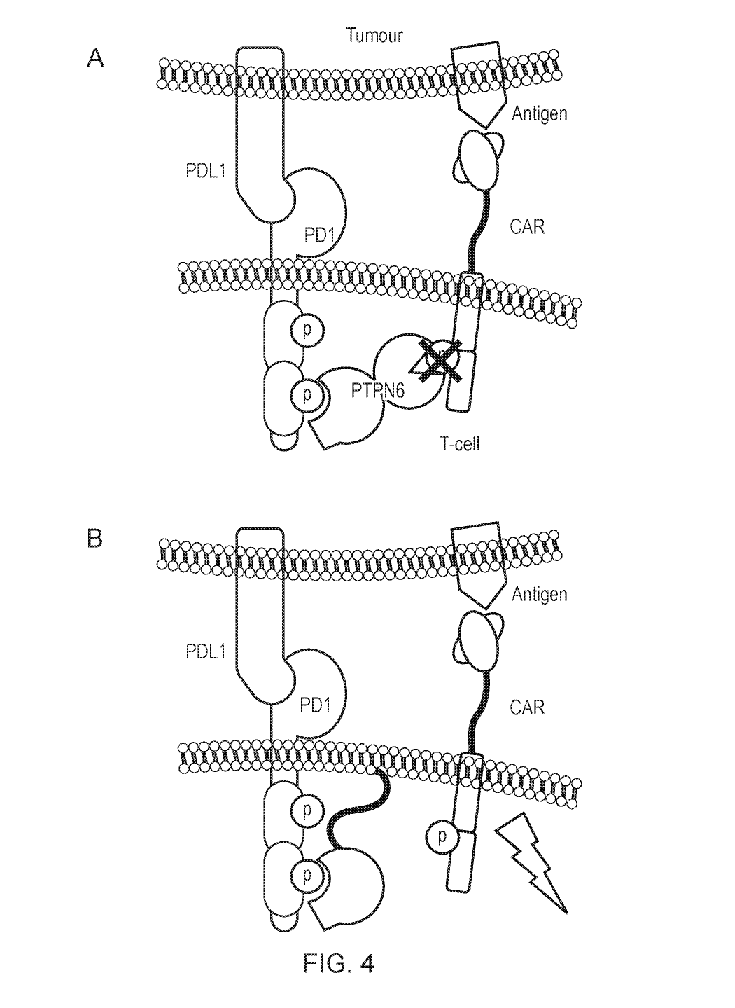

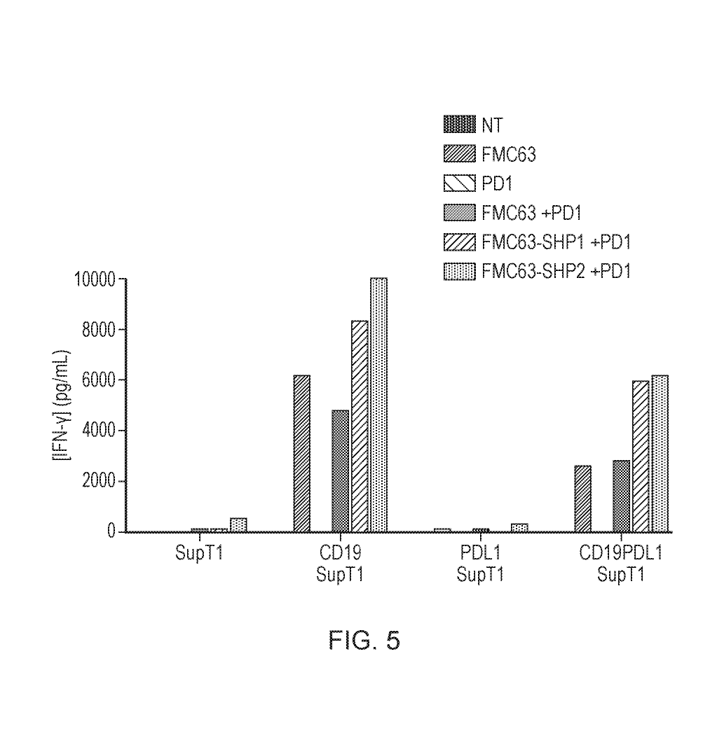

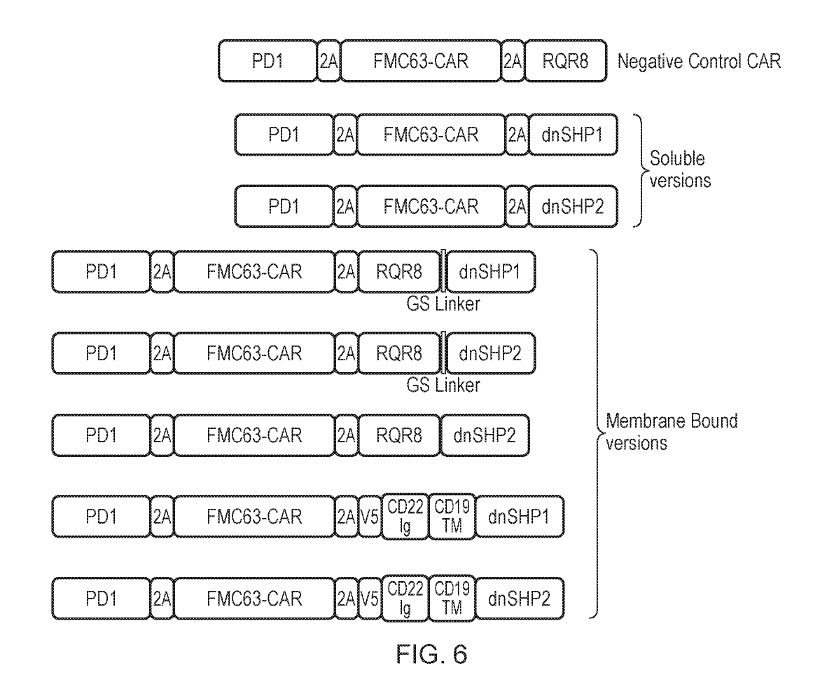

The pathway for T-cell inhibition by molecules such as PD1 is shown schematically in FIG. 1. The class of inhibitory receptors of which PD1 is a member contain an inhibitory motif known as an ITIM (Immune Tyrosine Inhibitory Motif). Upon recognition of their ligand, ITIMs become phosphorylated by membrane bound kinases such as Ick. Upon phosphorylation, these ITIMs are then recognized by one of only two SH2 proteins: SHP-1 and SHP-2. These proteins are unique in the class of SH2 containing proteins in that they contain a phosphatase. Upon recruitment to the ITIM, this phosphatase dephosphorylates key residues associated with T-cell activation.

The present inventors have shown that expression of a form of SHP-1 and SHP-2 which lacks a functional phosphatase domain can block CAR T-cell inhibition.

This approach has several advantages: it is simple; does not cause systemic toxicity;

and most importantly blocks a broad range of inhibitory pathways.

The present inventors have also found that it is possible to enhance the effect by “concentrating” the STMP at the cell membrane, at the site of the immunological synapse.

In a first aspect, the present invention provides a signal transduction modifying protein which comprises:

(i) a domain which binds a phosphorylated immunoreceptor tyrosine-based inhibition motif (pITIM); and

(ii) a membrane localisation domain.

The membrane localisation domain may comprise a myristoyl group, a palmitoyl group and/or a prenyl group.

The membrane localisation domain may bind to an entity which, inside a cell, is positioned in or near the membrane.

The membrane localisation domain may bind CD4 or CD8

The membrane localisation domain may be or comprise the amino acid shown as SEQ ID No. 13, or a part thereof which causes membrane localisation of the STMP when expressed inside a cell.

The membrane localisation domain may be or comprise a transmembrane domain.

The pITIM-binding domain may comprise an SH2 domain, such as an SH2 domain from SHP-1 and/or SHP-2 SH2.

The signal transduction modifying protein may lack a functional phosphatase domain.

The phosphatase domain may be partially or completely deleted.

The signal transduction modifying protein may comprise an inactivated phosphatase domain.

The phosphatase domain may comprise one or more amino acid mutations compared to a wild-type phosphatase domain, rendering it non-functional. The mutation may, for example involve deleting or replacing one or more cysteine residues. The mutation may be a cysteine-serine substitution.

The phosphatase domain may be a non-functional SHP-1 phosphatase domain, for example a mutated SHP-1 phosphatase domain comprising the sequence shown as SEQ ID No. 11.

The phosphatase domain may be a non-functional SHP-2 phosphatase domain, for example a mutated SHP-2 phosphatase domain comprising the sequence shown as SEQ ID No. 12.

In a second aspect, the present invention provides a cell which comprises a signal transduction modifying protein according to the first aspect of the invention.

The cell according may comprises two signal modifying proteins as defined above; wherein the pITIM-binding domain of the first signal transduction modifying protein comprises a SHP-1 SH2 domain; and the pITIM-binding domain of the second signal transduction modifying protein comprises a SHP-2 SH2 domain.

The cell may also comprise a chimeric antigen receptor (CAR).

In a third aspect, the present invention provides a nucleic acid sequence which encodes a signal transduction modifying protein according to the first aspect of the invention.

In a fourth aspect, the present invention provides a nucleic acid construct which comprises:
i) a first nucleic acid sequence according to the third aspect of the invention; and
ii) a second nucleic acid sequence which encodes a chimeric antigen receptor (CAR).

In a fifth aspect, the present invention provides a vector which comprises a nucleic acid sequence according to the third aspect of the invention or a nucleic acid construct according to the fourth aspect of the invention.

In a sixth aspect, there is provided a pharmaceutical composition comprising a plurality of cells according to the second aspect of the invention.

In a seventh aspect, there is provided a pharmaceutical composition according to the sixth aspect of the invention for use in treating and/or preventing a disease.

In an eighth aspect, there is provided a method for treating and/or preventing a disease, which comprises the step of administering a pharmaceutical composition according to the sixth aspect of the invention to a subject.

The method may also comprise the step of administering an immune checkpoint inhibitor to the subject, which immune checkpoint inhibitor inhibits a non-ITIM-mediated pathway. For example, the immune checkpoint inhibitor may be or comprise a CTLA4 pathway inhibitor such as a CTLA4 antibody.

The combination of systemic CTLA4 blockade along with CAR T-cells expressing an STMP which blocks SHP-1 and/or SHP-2 means that the CAR T-cell is resistant to two classes of inhibitory signals. It also means that local T-cells would be released from CTLA4 inhibition and which may give tolerable levels of systemic toxicity.

The method may comprise the following steps:

(i) isolation of a cell containing sample from a subject;

(ii) transduction or transfection of the cells with a nucleic acid sequence according to the third aspect of the invention; a nucleic acid construct according to the fourth aspect of the invention; or a vector according to the fifth aspect of the invention; and

(iii) administration the cells from (ii) to the subject.

In a ninth aspect, there is provided the use of a pharmaceutical composition according to the sixth aspect of the invention in the manufacture of a medicament for the treatment and/or prevention of a disease.

The disease may be cancer. The cancer may be a solid tumour.

In a tenth aspect there is provided a method for making a cell according to the second aspect of the invention, which comprises the step of introducing: a nucleic acid sequence according to the third aspect of the invention; a nucleic acid construct according to the fourth aspect of the invention; or a vector according to the fifth aspect of the invention into the cell.

The cell may be from a sample isolated from a subject.

### DETAILED DESCRIPTION

Signal Transduction Modifying Protein

The present invention relates to a signal transduction modifying protein (STMP). The STMP may modulate signal transduction in a T-cell. For example, it may reduce or block the inhibition of T-cell activation by ITIM-containing molecules such as PD1.

The STMP of the present invention comprises a domain which binds a phosphorylated immunoreceptor tyrosine-based inhibition motif (pITIM)

The STMP of the present invention lacks a functional phosphatase domain. The STMP may not comprise a phosphatase or it may comprise a partially or completely inactive phosphatase. The phosphatase may be inactivated by, for example, truncation or mutation of one or more amino acids.

pITIM-Binding Domain

The STMP of the present invention comprises a domain which binds a phosphorylated immunoreceptor tyrosine-based inhibition motif (pITIM).

An ITIM is a conserved sequence of amino acids (S/I/V/LxYxxI/V/L) that is found in the cytoplasmic tails of many inhibitory receptors of the immune system. After ITIM-possessing inhibitory receptors interact with their ligand, their ITIM motif becomes phosphorylated by enzymes of the Src kinases.

Immune inhibitory receptors such as PD1, PDCD1, BTLA4, LILRB1, LAIR1, CTLA4, the Killer inhibitory receptor family (KIR) including KIR2DL1, KIR2DL4, KIR2DL5, KIR3DL1 and KIR3DL3 contain ITIMs. The pITIM binding domain of the STMP of the present invention may bind a phosphorylated ITIM from one or more of these proteins.

The pITIM-binding domain may comprise an SH2 domain.

Src Homology 2 (SH2) Domain

Intracellular signalling pathways are initiated and controlled by the reversible post-translational modification of proteins including phosphorylation, ubiquitinylation and acetylation.

SH2 domains are modular protein domains that serve as adaptors and mediate protein-protein interactions by binding to phosphorylated peptides in their respective protein binding partners, often cell surface receptors. SH2 domains typically bind a phosphorylated tyrosine residue in the context of a longer peptide motif within a target protein, and SH2 domains represent the largest class of known pTyr-recognition domains.

Although SH2 domains lack any intrinsic catalytic activity they are frequently coupled to independent catalytic domains and thus, in response to a specific input signal, serve to localize these catalytic domains so particular sub-cellular locations or to the vicinity of appropriate substrates, activators or inhibitors. In addition SH2 domains can also be found linked to adaptor protein domains and so can serve in the formation of large multi-protein complexes.

The STMP protein of the present invention may comprise one or more SH2 domains from SHP-1 and/or SHP-2.

SRC Homology Region 2 Domain-Containing Phosphatase-1 (SHP-1)

SHP-1 is also known as tyrosine-protein phosphatase non-receptor type 6 (PTPN6). It is a member of the protein tyrosine phosphatase family.

The N-terminal region of SHP-1 contains two tandem SH2 domains which mediate the interaction of SHP-1 and its substrates. The C-terminal region contains a tyrosine-protein phosphatase domain.

SHP-1 is capable of binding to, and propagating signals from, a number of inhibitory immune receptors or ITIM containing receptors. Examples of such receptors include, but are not limited to, PD1, PDCD1, BTLA4, LILRB1, LAIR1, CTLA4, KIR2DL1, KIR2DL4, KIR2DL5, KIR3DL1 and KIR3DL3.

Human SHP-1 protein has the UniProtKB accession number P29350. This sequence is 595 amino acids in length and is shown as SEQ ID NO: 1.

SHP-1 amino acid sequence
(SEQ ID NO: 1)
MVRWFHRDLSGLDAETLLKGRGVHGSFLARPSRKNQGDFSLSVRVGD
QVTHIRIQNSGDFYDLYGGEKFATLTELVEYYTQQQGVLQDRDGTII
HLKYPLNCSDPTSERWYHGHMSGGQAETLLQAKGEPWTFLVRESLSQ
PGDFVLSVLSDQPKAGPGSPLRVTHIKVMCEGGRYTVGGLETFDSLT
DLVEHFKKTGIEEASGAFVYLRQPYYATRVNAADIENRVLELNKKQE
SEDTAKAGFWEEFESLQKQEVKNLHQRLEGQRPENKGKNRYKNILPF
DHSRVILQGRDSNIPGSDYINANYIKNQLLGPDENAKTYIASQGCLE
ATVNDFWQMAWQENSRVIVMTTREVEKGRNKCVPYWPEVGMQRAYGP
YSVTNCGEHDTTEYKLRTLQVSPLDNGDLIREIWHYQYLSWPDHGVP
SEPGGVLSFLDQINQRQESLPHAGPIIVHCSAGIGRTGTIIVIDMLM
ENISTKGLDCDIDIQKTIQMVRAQRSGMVQTEAQYKFIYVAIAQFIE
TTKKKLEVLQSQKGQESEYGNITYPPAMKNAHAKASRTSSKHKEDVY
ENLHTKNKREEKVKKQRSADKEKSKGSLKRK

There are also three alternative isoforms of SHP-1, as shown in the following table:

| | Amino | | |
|---|---|---|---|
| | acid | | Overall |
| | position | Difference from SEQ ID No. 1 | length |
| Isoform 2 | 1-3 | MVR → MLSRG | 597 |
| Isoform 3 | 1-39 | Missing | 556 |
| | 40-44 | SLSVR → MLSRG | |
| Isoform 4 | 559-595 | HKEDVYENLH . . . EKSKGSLKRK → | 624 |
| Also | | SLESSAGTVA . . . CTLRTRGRRK | |
| known as | | | |
| SHP-1L | | | |

The STMP of the invention may comprise or consist of a SHP-1 SH2 domain. In this respect, the STMP may comprise or consist of the sequence shown as SEQ ID NO: 2.

SHP-1 SH2 complete domain
(SEQ ID NO: 2)
MVRWFHRDLSGLDAETLLKGRGVHGSFLARPSRKNQGDFSLSVRVGD
QVTHIRIQNSGDFYDLYGGEKFATLTELVEYYTQQQGVLQDRDGTII
HLKYPLNCSDPTSERWYHGHMSGGQAETLLQAKGEPWTFLVRESLSQ
PGDFVLSVLSDQPKAGPGSPLRVTHIKVMCEGGRYTVGGLETFDSLT
DLVEHFKKTGIEEASGAFVYLRQPYY

SHP-1 has two SH2 domains at the N-terminal end of the sequence, at residues 4-100 and 110-213 of the sequence shown as SEQ ID No. 2. The STMP of the invention may therefore comprise one or both of the sequences shown as SEQ ID No. 3 and 4.

SHP-1 SH2 1
(SEQ ID NO: 3)
WFHRDLSGLDAETLLKGRGVHGSFLARPSRKNQGDFSLSVRVGDQVT
HIRIQNSGDFYDLYGGEKFATLTELVEYYTQQQGVLQDRDGTIIHLK
YPL
SHP-1 SH2 2
(SEQ ID No. 4)
WYHGHMSGGQAETLLQAKGEPWTFLVRESLSQPGDFVLSVLSDQPKA
GPGSPLRVTHIKVMCEGGRYTVGGLETFDSLTDLVEHFKKTGIEEAS
GAFVYLRQPY

The STMP may comprise a variant of SEQ ID NO: 2, 3 or 4 having at least 80, 85, 90, 95, 98 or 99% sequence identity, provided that the variant sequence is a SH2 domain sequence capable of binding a pITIM domain. For example, the variant sequence may be capable of binding to the phosphorylated tyrosine residues in the cytoplasmic tail of PD1, PDCD1, BTLA4, LILRB1, LAIR1, CTLA4, KIR2DL1, KIR2DL4, KIR2DL5, KIR3DL1 or KIR3DL3. The variant sequence may be the equivalent sequence to of SEQ ID NO: 2, 3 or 4 when derived from isoform 2, 3 or 4 of SHP-1.

SHP-2

SHP-2, also known as PTPN11, PTP-1D and PTP-2C, is also a member of the protein tyrosine phosphatase (PTP) family. Like SHP-1, SHP-2 has a domain structure that consists of two tandem SH2 domains in its N-terminus followed by a protein tyrosine phosphatase (PTP) domain. In the inactive state, the N-terminal SH2 domain binds the PTP domain and blocks access of potential substrates to the active site. Thus, SHP-2 is auto-inhibited. Upon binding to target phospho-tyrosyl residues, the N-terminal SH2 domain is released from the PTP domain, catalytically activating the enzyme by relieving the auto-inhibition.

Human SHP-2 has the UniProtKB accession number P35235-1. This sequence is 597 amino acids in length and is shown as SEQ ID NO: 5.

SHP-2 amino acid sequence
(SEQ ID NO: 5)
MTSRRWFHPNITGVEAENLLLTRGVDGSFLARPSKSNPGDFTLSVRR
NGAVTHIKIQNTGDYYDLYGGEKFATLAELVQYYMEHHGQLKEKNGD
VIELKYPLNCADPTSERWFHGHLSGKEAEKLLTEKGKHGSFLVRESQ
SHPGDFVLSVRTGDDKGESNDGKSKVTHVMIRCQELKYDVGGGERFD
SLTDLVEHYKKNPMVETLGTVLQLKQPLNTTRINAAEIESRVRELSK
LAETTDKVKQGFWEEFETLQQQECKLLYSRKEGQRQENKNKNRYKNI
LPFDHTRVVLHDGDPNEPVSDYINANIIMPEFETKCNNSKPKKSYIA
TQGCLQNTVNDFWRMVFQENSRVIVMTTKEVERGKSKCVKYWPDEYA
LKEYGVMRVRNVKESAAHDYTLRELKLSKVGQALLQGNTERTVWQYH
FRTWPDHGVPSDPGGVLDFLEEVHHKQESIVDAGPVVVHCSAGIGRT
GTFIVIDILIDIIREKGVDCDIDVPKTIQMVRSQRSGMVQTEAQYRF
IYMAVQHYIETLQRRIEEEQKSKRKGHEYTNIKYSLVDQTSGDQSPL
PPCTPTPPCAEMREDSARVYENVGLMQQQRSFR

There are also two alternative isoforms of SHP-2, as shown in the following table:

| | Amino | | |
|---|---|---|---|
| | acid | Difference from SEQ ID | |
| | position | No. 5 | Overall length |
| Isoform 2 | 408-411 | Missing | 593 |
| Isoform 3 | 408-411 | Missing | 460 |
| | 464 | S → R | |
| | 465-597 | Missing | |

The STMP of the invention may comprise or consist of a SHP-2 SH2 domain. In this respect, the STMP may comprise or consist of the first SH2 domain of SHP-2, for example comprising amino acids 6-102 of SEQ ID NO. 5 or the second SH2 domain of SHP-2, for example comprising amino acids 112-216 of SHP-2. The STMP may comprise or consist of the sequence shown as SEQ ID NO: 6, 7 or 8. The STMP may comprise a variant of SEQ ID NO: 6, 7 or 8 having at least 80, 85, 90, 95, 98 or 99% sequence identity, provided that the variant sequence is a SH2 domain sequence capable of binding a pITIM domain. For example, the variant sequence may be capable of binding to the phosphorylated tyrosine residues in the cytoplasmic tail of PD1, PDCD1, BTLA4, LILRB1, LAIR1, CTLA4, KIR2DL1, KIR2DL4, KIR2DL5, KIR3DL1 or KIR3DL3. The variant sequence may be the equivalent sequence to of SEQ ID NO: 6, 7 or 8 when derived from isoform 2 or 3 of SHP-2.

SHP-2 first SH2 domain
(SEQ ID NO: 6)
WFHPNITGVEAENLLLTRGVDGSFLARPSKSNPGDFTLSVRRNGAVT
HIKIQNTGDYYDLYGGEKFATLAELVQYYMEHHGQLKEKNGDVIELK
YPL
SHP-2 second SH2 domain
(SEQ ID No. 7)
WFHGHLSGKEAEKLLTEKGKHGSFLVRESQSHPGDFVLSVRTGDDKG
ESNDGKSKVTHVMIRCQELKYDVGGGERFDSLTDLVEHYKKNPMVET
LGTVLQLKQPL
SHP-2 both SH2 domains
(SEQ ID No. 8)
WFHPNITGVEAENLLLTRGVDGSFLARPSKSNPGDFTLSVRRNGAVT
HIKIQNTGDYYDLYGGEKFATLAELVQYYMEHHGQLKEKNGDVIELK
YPLNCADPTSERWFHGHLSGKEAEKLLTEKGKHGSFLVRESQSHPGD
FVLSVRTGDDKGESNDGKSKVTHVMIRCQELKYDVGGGERFDSLTDL
VEHYKKNPMVETLGTVLQLKQPL

Phosphatase Domain

The sequence of human SHP-1 phosphatase and SHP-2 phosphatase domains are shown as SEQ ID NO: 9 and 10 respectively.

SEQ ID NO: 9-SHP-1 phosphatase domain
FWEEFESLQKQEVKNLHQRLEGQRPENKGKNRYKNILPFDHSRVILQ
GRDSNIPGSDYINANYIKNQLLGPDENAKTYIASQGCLEATVNDFWQ
MAWQENSRVIVMTTREVEKGRNKCVPYWPEVGMQRAYGPYSVTNCGE
HDTTEYKLRTLQVSPLDNGDLIREIWHYQYLSWPDHGVPSEPGGVLS
FLDQINQRQESLPHAGPIIVHCSAGIGRTGTIIVIDMLMENISTKGL
DCDIDIQKTIQMVRAQRSGMVQTEAQYKFIYVAIAQF
SEQ ID NO: 10-SHP-2 phosphatase domain
FWEEFETLQQQECKLLYSRKEGQRQENKNKNRYKNILPFDHTRVVLH
DGDPNEPVSDYINANIIMPEFETKCNNSKPKKSYIATQGCLQNTVND
FWRMVFQENSRVIVMTTKEVERGKSKCVKYWPDEYALKEYGVMRVRN
VKESAAHDYTLRELKLSKVGQALLQGNTERTVWQYHFRTWPDHGVPS
DPGGVLDFLEEVHHKQESIMDAGPVVVHCSAGIGRTGTFIVIDILID
IIREKGVDCDIDVPKTIQMVRSQRSGMVQTEAQYRFIYMA

The STMP of the present invention may lack a phosphatase domain or comprise a non-functional phosphatase domain. For example it may comprise a partially deleted phosphatase domain or an inactivated phosphatase domain

Truncated Phosphatase Domain

The STMP of the present invention may completely lack a phosphatase domain. For example, the STMP may comprise one or both SHP-1/SHP-2 SH2 domains, but be truncated to remove the SHP-1/SHP-2 phosphatase.

Alternatively, the STMP of the present invention may comprise a partially truncated phosphatase which comprises part of a phosphatase, for example a portion of the sequence shown as SEQ ID No. 9 or 10, provided that the truncated phosphatase has reduced capacity to dephosphorylate downstream proteins compared to the wild-type phosphatase from which it was derived. The truncated phosphatase may have effectively no residual phosphatase activity.

Inactivated Phosphatase Domain

The STMP of the present invention may comprise a phosphatase domain, for example an SHP-1 or SHP-2 phosphatase or derivative thereof, which is inactivated so that it has reduced or no capacity to dephosphorylate ITAM-containing proteins.

The phosphatase may, for example, comprise one or more amino acid mutations such that it has reduced phosphatase activity compared to the wild-type sequence.

The mutation may, for example, be an addition, deletion or substitution.

The mutation may comprise the deletion or substitution of one or more cysteine residues.

The variant phosphatase sequence may have a mutation to cysteine 210 with reference to the sequence shown as SEQ ID No. 9. This is position 453 in the full length SHP-1 sequence (isoform 1). The mutation may be a cysteine to serine substitution. A variant sequence having a C210S substitution is shown as SEQ ID No. 11 (the C210S substitution is shown in bold and underlined).

SEQ ID No. 11
FWEEFESLQKQEVKNLHQRLEGQRPENKGKNRYKNILPFDHSRVILQ
GRDSNIPGSDYINANYIKNQLLGPDENAKTYIASQGCLEATVNDFWQ
MAWQENSRVIVMTTREVEKGRNKCVPYWPEVGMQRAYGPYSVTNCGE
HDTTEYKLRTLQVSPLDNGDLIREIWHYQYLSWPDHGVPSEPGGVLS
FLDQINQRQESLPHAGPIIVHSSAGIGRTGTIIVIDMLMENISTKGL
DCDIDIQKTIQMVRAQRSGMVQTEAQYKFIYVAIAQF

The variant phosphatase sequence may have a mutation to cysteine 217 with reference to the sequence shown as SEQ ID No. 10. This is position 463 in the full length SHP-2 sequence (isoform 1). The mutation may be a cysteine to serine substitution. A variant sequence having a C217S substitution is shown as SEQ ID No. 12 (the C217S substitution is shown in bold and underlined).

SEQ ID No. 12
FWEEFETLQQQECKLLYSRKEGQRQENKNKNRYKNILPFDHTRVVLH
DGDPNEPVSDYINANIIMPEFETKCNNSKPKKSYIATQGCLQNTVND
FWRMVFQENSRVIVMTTKEVERGKSKCVKYWPDEYALKEYGVMRVRN
VKESAAHDYTLRELKLSKVGQALLQGNTERTVWQYHFRTWPDHGVPS
DPGGVLDFLEEVHHKQESIMDAGPVVVHSSAGIGRTGTFIVIDILID
IIREKGVDCDIDVPKTIQMVRSQRSGMVQTEAQYRFIYMA

Membrane Localisation Domain

The STMP of the present invention may comprise a membrane localisation domain. The membrane localisation domain may cause the STMP to be “concentrated” at or close to the cell membrane.

The membrane localisation domain may be or comprise a membrane tether. The membrane tether may be any sequence, signal or domain which is capable of localising the transcription factor and protease recognition site proximal to a membrane. For example, the membrane tether may be a myrsitylation signal or a transmembrane domain.

A transmembrane domain may be any protein structure which is thermodynamically stable in a membrane. This is typically an alpha helix comprising of several hydrophobic residues. The transmembrane domain of any transmembrane protein can be used to supply the transmembrane portion. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may be derived from CD28, which gives good stability.

Alternatively the membrane localisation domain may direct the STMP to a protein or other entity which is located at the cell membrane, for example by binding the membrane-proximal entity. The STMP may, for example, comprise a domain which binds a molecule which is involved in the immune synapse, such as TCR/CD3, CD4 or CD8.

Myristoylation

Myristoylation is a lipidation modification where a myristoyl group, derived from myristic acid, is covalently attached by an amide bond to the alpha-amino group of an N-terminal glycine residue. Myristic acid is a 14-carbon saturated fatty acid also known as n-Tetradecanoic acid. The modification can be added either co-translationally or post-translationally. N-myristoyltransferase (NMT) catalyzes the myristic acid addition reaction in the cytoplasm of cells. Myristoylation causes membrane targeting of the protein to which it is attached, as the hydrophobic myristoyl group interacts with the phospholipids in the cell membrane.

The STMP of the present invention may comprise a sequence capable of being myristoylated by a NMT enzyme. The STMP of the present invention may comprise a myristoyl group when expressed in a cell.

The STMP may comprise a consensus sequence such as: NH2-G1-X2-X3-X4-S5-X6-X7-X8 which is recognised by NMT enzymes.

Palmitoylation

Palmitoylation is the covalent attachment of fatty acids, such as palmitic acid, to cysteine and less frequently to serine and threonine residues of proteins. Palmitoylation enhances the hydrophobicity of proteins and can be used to induce membrane association. In contrast to prenylation and myristoylation, palmitoylation is usually reversible (because the bond between palmitic acid and protein is often a thioester bond). The reverse reaction is catalysed by palmitoyl protein thioesterases.

In signal transduction via G protein, palmitoylation of the a subunit, prenylation of the γ subunit, and myristoylation is involved in tethering the G protein to the inner surface of the plasma membrane so that the G protein can interact with its receptor.

The STMP of the present invention may comprise a sequence capable of being palmitoylated. The STMP of the present invention may comprise additional fatty acids when expressed in a cell which causes membrane localisation.

Prenylation

Prenylation (also known as isoprenylation or lipidation) is the addition of hydrophobic molecules to a protein or chemical compound. Prenyl groups (3-methyl-but-2-en-1-yl) facilitate attachment to cell membranes, similar to lipid anchors like the GPI anchor.

Protein prenylation involves the transfer of either a farnesyl or a geranyl-geranyl moiety to C-terminal cysteine(s) of the target protein. There are three enzymes that carry out prenylation in the cell, farnesyl transferase, Caax protease and geranylgeranyl transferase I.

The STMP of the present invention may comprise a sequence capable of being prenylated. The STMP of the present invention may comprise one or more prenyl groups when expressed in a cell which causes membrane localisation.

CD4/CD8 Binding Sequence

The membrane localisation sequence of an STMP of the invention may bind to an entity, such as a protein, which is positioned at, in or near the membrane of a cell.

For example, the membrane localisation sequence may bind to CD4 or CD8.

The amino terminal sequence of Ick, which is shown as SEQ ID No. 13 comprises three different types of membrane localisation sequence, as follows:
an N-myristoyl glycine, which is a substrate for myristoylation (highlighted in grey)
two S-palmitoyl cysteine, which are substrates for palmitoylation (shown in bold and italics), and
two cysteine residues involved in a zinc clasp necessary for CD4/CD8 binding (underlined).

SEQ ID No. 13

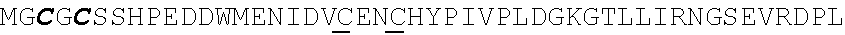

VTYEGSNPPASPLQDNLVIALHSY

The membrane localisation sequence of the STMP of the present invention may comprise any one or more of the myristoylation sequence, the palmitoylation sequence and the CD4/CD8 binding sequence from SEQ ID No. 13. The membrane localisation domain may comprise or consist of SEQ ID No. 13 or a variant thereof having at least 70%, 80%, 90% or 95% amino acid identity, which retains the capacity to localise the STMP at the cell membrane.

The membrane localisation sequence comprising or consisting of SEQ ID No. 13 or a variant thereof may be positioned at the N-terminal end of the STMP sequence, in from of the pITIM-binding domain.

Nucleic Acid

In one aspect the present invention provides a nucleic acid which encodes a STMP according to the present invention.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Nucleic Acid Construct

In one aspect the present invention provides a nucleic acid construct which co-expresses a STMP of the present invention with another protein. The nucleic acid construct may comprise: a nucleic acid sequence encoding a STMP of the present invention; and a nucleic acid encoding another protein.

The present invention provides a nucleic acid construct which co-expresses a STMP of the present invention with a chimeric antigen receptor. The nucleic acid construct may comprise: (i) a nucleic acid sequence encoding a STMP of the present invention; and (ii) a nucleic acid encoding a chimeric antigen receptor.

The chimeric antigen receptor (CAR) may be an activatory CAR comprising an ITAM-containing endodomain, such as CD3 zeta.

The nucleic acid construct may also comprise a nucleic acid sequence enabling expression of two or more proteins. For example, it may comprise a sequence encoding a cleavage site between the two nucleic acid sequences. The cleavage site may be self-cleaving, such that when the nascent polypeptide is produced, it is immediately cleaved into the two proteins without the need for any external cleavage activity.

Various self-cleaving sites are known, including the Foot-and-Mouth disease virus (FMDV) 2a self-cleaving peptide, which has the sequence:

SEQ ID NO: 14
RAEGRGSLLTCGDVEENPGP
or
SEQ ID NO: 15
QCTNYALLKLAGDVESNPGP

The co-expressing sequence may alternatively be an internal ribosome entry sequence (IRES) or an internal promoter.

Chimeric Antigen Receptor (CAR)

A classical chimeric antigen receptor (CAR) is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41 BB which transmit survival signals. Even more potent third generation CARs have now been described which have endodomains capable of transmitting activation, proliferation and survival signals.

CAR-encoding nucleic acids may be transferred to T cells using, for example, retroviral vectors. Lentiviral vectors may be employed. In this way, a large number of cancer-specific T cells can be generated for adoptive cell transfer. When the CAR binds the target-antigen, this results in the transmission of an activating signal to the T-cell it is expressed on. Thus the CAR directs the specificity and cytotoxicity of the T cell towards tumour cells expressing the targeted antigen.

CARs typically therefore comprise: (i) an antigen-binding domain; (ii) a spacer; (iii) a transmembrane domain; and (iii) an intracellular domain which comprises or associates with a signalling domain.

Antigen Binding Domain

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain may comprise a domain which is not based on the antigen binding site of an antibody. For example the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell.

The antigen binding domain may be based on a natural ligand of the antigen.

The antigen binding domain may comprise an affinity peptide from a combinatorial library or a de novo designed affinity protein/peptide.

Spacer Domain

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

Transmembrane Domain

The transmembrane domain is the sequence of the CAR that spans the membrane.

A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may be derived from CD28, which gives good receptor stability.

Activating Endodomain

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

Where a CAR comprises an activating endodomain, it may comprise the CD3-Zeta endodomain alone, the CD3-Zeta endodomain with that of either CD28 or OX40 or the CD28 endodomain and OX40 and CD3-Zeta endodomain.

Any endodomain which contains an ITAM motif can act as an activation endodomain.

Vector

The present invention also provides a vector, or kit of vectors which comprises one or more nucleic acid sequence(s) or construct(s) according to the present invention. Such a vector may be used to introduce the nucleic acid sequence(s) or construct(s) into a host cell so that it expresses the proteins encoded by the nucleic acid sequence or construct.

The vector may, for example, be a plasmid or a viral vector, such as a retroviral vector or a lentiviral vector, or a transposon based vector or synthetic mRNA.

The vector may be capable of transfecting or transducing a T cell.

Cell

The present invention also relates to a cell, such as an immune cell, which comprises a STMP, nucleic acid and/or nucleic acid construct of the present invention.

The cell may be a cytolytic immune cell.

Cytolytic immune cells can be T cells or T lymphocytes which are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Natural Killer Cells (or NK cells) are a type of cytolytic cell which form part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Cells expressing an STMP of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, the cells may be derived from ex vivo differentiation of inducible progenitor cells or embryonic progenitor cells to, for example, T cells. Alternatively, an immortalized cell line which retains its lytic function and could act as a therapeutic may be used.

In all these embodiments, cells are generated by introducing DNA or RNA coding for the receptor component and signalling component by one of many means including transduction with a viral vector, transfection with DNA or RNA.

The cell of the invention may be an ex vivo cell from a subject. The cell may be from a peripheral blood mononuclear cell (PBMC) sample. Cells may be activated and/or expanded prior to being transduced with nucleic acid sequence or construct of the invention, for example by treatment with an anti-CD3 monoclonal antibody.

The cell of the invention may be made by:
(i) isolation of a cell-containing sample from a subject or other sources listed above; and
(ii) transduction or transfection of the cells with a nucleic acid sequence or construct according to the invention.

Composition

The present invention also relates to a pharmaceutical composition containing a plurality of cells of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

The pharmaceutical composition may also comprise an immune checkpoint inhibitor which inhibits a non-ITIM-mediated pathway (see next section).

Method of Treatment

The cells of the present invention may be capable of killing target cells, such as cancer cells.

The cells of the present invention may be used for the treatment of an infection, such as a viral infection.

The cells of the invention may also be used for the control of pathogenic immune responses, for example in autoimmune diseases, allergies and graft-vs-host rejection.

The cells of the invention may be used for the treatment of a cancerous disease, such as bladder cancer, breast cancer, colon cancer, endometrial cancer, kidney cancer (renal cell), leukemia, lung cancer, melanoma, non-Hodgkin lymphoma, pancreatic cancer, prostate cancer and thyroid cancer.

The cells of the invention may be used to treat: cancers of the oral cavity and pharynx which includes cancer of the tongue, mouth and pharynx; cancers of the digestive system which includes oesophageal, gastric and colorectal cancers; cancers of the liver and biliary tree which includes hepatocellular carcinomas and cholangiocarcinomas; cancers of the respiratory system which includes bronchogenic cancers and cancers of the larynx; cancers of bone and joints which includes osteosarcoma; cancers of the skin which includes melanoma; breast cancer; cancers of the genital tract which include uterine, ovarian and cervical cancer in women, prostate and testicular cancer in men; cancers of the renal tract which include renal cell carcinoma and transitional cell carcinomas of the utterers or bladder; brain cancers including gliomas, glioblastoma multiforme and medullobastomas; cancers of the endocrine system including thyroid cancer, adrenal carcinoma and cancers associated with multiple endocrine neoplasm syndromes; lymphomas including Hodgkin's lymphoma and non-Hodgkin lymphoma; Multiple Myeloma and plasmacytomas; leukaemias both acute and chronic, myeloid or lymphoid; and cancers of other and unspecified sites including neuroblastoma.

Treatment with the cells of the invention may help prevent the escape or release of tumour cells which often occurs with standard approaches.

The method may comprise the step of administering an immune checkpoint inhibitor to the subject, which immune checkpoint inhibitor inhibits a non-ITIM-mediated pathway. Non-ITIM mediated means that the pathway does not involve an ITIM-containing protein such as PD1, PDCD1, BTLA4, LILRB1, LAIR1, CTLA4, KIR2DL1, KIR2DL4, KIR2DL5, KIR3DL1 or KIR3DL3.

An ITIM-mediated pathway may be mediated by SHP-1 and/or SHP-2; whereas SHP-1 and SHP-2 are not involved in a non-ITIM-mediated pathway.

The immune checkpoint inhibitor may inhibit the CTLA4 pathway. CTLA-4 binds CD80 (also known as B7-1) and CD86 (also known as B7-2) with greater affinity and avidity than CD28 thus enabling it to outcompete CD28 for its ligands. CTLA4 transmits an inhibitory signal to T cells whereas CD28 transmits a stimulatory signal.

The CTLA4 pathway inhibitor may be a CTLA4 antibody such as ipilimumab or tremelimumab.

Alternatively the immune checkpoint inhibitor may inhibit another non-ITIM-mediated pathway, such as the pathway mediated by one of the following molecules: TIM3, KIR, LAGS, ICOS and VISTA.

The present invention also provides a kit which comprises:
(i) a plurality of cells according to the invention; and
(ii) an immune checkpoint inhibitor which inhibits a non-ITIM-mediated pathway
   for separate, subsequent or simultaneous administration to a subject.

The invention will now be further described by way of Examples, which are meant to serve to assist one of ordinary skill in the art in carrying out the invention and are not intended in any way to limit the scope of the invention.

### EXAMPLES

### Example 1—PD-1 Signal Blockade Using Truncated SHP-1 (PTPN6) or Truncated SHP-2

PBMC cells were transduced as shown in the following table:

| Name on | | |
|---|---|---|
| FIG. 15 key | Description | Construct(s) |
| NT | Untransduced | — |
| FMC63 | Transduced with CD19 CAR only | SFG.aCD19_fmc63-HCH2CH3w- |
| | | CD28tmZw |
| PD1 | Transduced with PD1 only | pDual-PD1-GFP |
| FMC63 + PD1 | Co-transduced with CD19CAR | SFG.aCD19_fmc63-HCH2CH3w- |
| | and PD1 | CD28tmZw and pDual-PD1-GFP |
| FMC63- | Co-transduced with a) bicistronic | SFG.aCD19_fmc63-HCH2CH3w- |
| SHP1 + PD1 | construct encoding CD19CAR | CD28tm-Zeta_w-2A-dualSH2_SHP-1 |
| | and truncated SHP1, and b) PD1 | and pDual-PD1-GFP |
| FMC63- | Co-transduced with a) bicistronic | SFG.aCD19_fmc63-HCH2CH3w- |
| SHP2 + PD1 | construct encoding CD19CAR | CD28tm-Zeta_w-2A-dualSH2_SHP-2 |
| | and truncated SHP1, and b) PD1 | and pDual-PD1-GFP |

The cells were co-cultured for 48 hours with SupT1 cells transduced with CD19, PDL1 or both and IFNγ release measured by ELISA. The results are shown in FIG. 5.

The presence of PDL1 on SupT1 target cells caused a reduction in IFNγ release. There was increased IFNγ release with PBMC which expressed CAR together with the truncated SHP-1 or truncated SHP-2 construct compared with those which expressed CAR alone. This indicates that the truncated SHP-1 and SHP-2 constructs successfully inhibited the PDL1 inhibitory signal from the target cells.

### Example 2—Investigating the Effect of Localising dnSHP1/SHP2 to the Membrane

Three different strategies are tested in order to localise dnSHP to the membrane:
(i) direct linkage to the sort-suicide gene RQR8;
(ii) linkage to the sort-suicide gene RQR8 either via a 48 bp G-S linker; and
(iii) linkage to the two extracellular Ig domains of the CD22 molecule (V5-tagged) and the CD19 molecule transmembrane domain

The constructs tested are shown in the following and in FIG. 6.

| | Construct Details |
|---|---|
| Control construct | SFG.PD1-2A-aCD19_fmc63-HCH2CH3w-CD28tm- |
| | Zeta_w-2A-RQR8 |
| Soluble SHP1 | SFG.PD1-2A-aCD19_fmc63-HCH2CH3w-CD28tm- |
| | Zeta_w-2A-dualSH2_SHP-1 |
| Soluble SHP2 | SFG.PD1-2A-aCD19_fmc63-HCH2CH3w-CD28tm- |
| | Zeta_w-2A-dualSH2_SHP-2 |
| Membrane bound | SFG.PD1-2A-aCD19_fmc63-HCH2CH3w-CD28tm- |
| SHP1 (via RQR8 + 48 bp | Zeta_w-2A-RQR8-16aaL-dualSH2_SHP-1 |
| G-S linker) | |
| Membrane bound | SFG.PD1-2A-aCD19_fmc63-HCH2CH3w-CD28tm- |
| SHP2 (via RQR8 + 48 bp | Zeta_w-2A-RQR8-16aaL-dualSH2_SHP-2 |
| G-S linker) | |
| Membrane bound | SFG.PD1-2A-aCD19_fmc63-HCH2CH3w-CD28tm- |
| SHP2 (via RQR8-no | Zeta_w-2A-RQR8-dualSH2_SHP-2 |
| linker) | |
| Membrane bound | SFG.PD1-2A-aCD19_fmc63-HCH2CH3w-CD28tm- |
| SHP1 (via 2 CD22 Ig | Zeta_w-2A-V5_tag-CD22_2Ig-CD19tm-RL-DN_SHP1 |
| domains + CD19TM) | |
| Membrane bound | SFG.PD1-2A-aCD19_fmc63-HCH2CH3w-CD28tm- |
| SHP2 (via 2 CD22 Ig | Zeta_w-2A-V5_tag-CD22_2Ig-CD19tm-RL-DN_SHP2 |
| domains + CD19TM) | |

PBMCs are transduced with the dnSHP soluble or membrane bound constructs after 24 h activation with CD3 and CD28 antibodies. Expression of the constructs is assessed 3 days post transduction upon staining with PD1-PE and either: hCD34-APC for the constructs that contain RQR8; or V5-APC for the constructs with the V5 tag. Transduced cells are depleted of the CD56+ (NK T cell) population and are subsequently maintained in culture.

Co-cultures for FACS based killing assay and ELISA are carried out at a 4:1 E:T ratio with a number of 2×10⁵ target cells, in 96 well plates. Target cell lines used are SupT1 cells expressing CD19 or expressing both CD19 and PDL1. Non transduced PBMCs (NT) are also cultured with the target cells at the same E:T ratio and are used as controls.

After 24 h incubation, supernatants are harvested to be used in subsequent IFN-gamma and IL-2 ELISAs. Cells are spun down, stained with 7AAD and CD3-PE-Cy7 and FACS analysed. Remaining target cells are enumerated as the live (7AAD negative), non T cell (CD3 negative) population.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A signal transduction modifying protein which comprises:
(i) a domain which binds a phosphorylated immunoreceptor tyrosine-based inhibition motif (pITIM); and
(ii) a membrane localisation domain.

2-6. (canceled)

7. A signal transduction modifying protein according to claim 1, wherein the pITIM-binding domain comprises a SHP-1 SH2 domain which lacks a functional phosphatase domain or a SHP-2 SH2 domain which lacks a functional phosphatase domain.

8-11. (canceled)

12. A signal transduction protein according to claim 7, in which the phosphatase domain is partially or completely deleted.

13. A signal transduction modifying protein according to claim 7, which comprises an inactivated phosphatase domain.

14. A signal transduction modifying protein according to claim 13, wherein the phosphatase domain comprises one or more amino acid mutations compared to a wild-type phosphatase domain, rendering it non-functional.

15-19. (canceled)

20. A cell which comprises a signal transduction modifying protein according to claim 1.

21. A cell according to claim 20 which comprises two signal modifying proteins; wherein the pITIM-binding domain of the first signal transduction modifying protein comprises a SHP-1 SH2 domain; and the pITIM-binding domain of the second signal transduction modifying protein comprises a SHP-2 SH2 domain.

22. A cell according to claim 20, which also comprises a chimeric antigen receptor (CAR).

23. A nucleic acid sequence which encodes a signal transduction modifying protein according to claim 1.

24. A nucleic acid construct which comprises:
i) a first nucleic acid sequence according to claim 23; and
ii) a second nucleic acid sequence which encodes a chimeric antigen receptor (CAR).

25. A vector which comprises a nucleic acid sequence according to claim 23 or a nucleic acid construct according to claim 24.

26. A pharmaceutical composition comprising a plurality of cells according to claim 20.

27. (canceled)

28. A method for treating and/or preventing a disease, which comprises the step of administering a pharmaceutical composition according to claim 26 to a subject.

29. A method according to claim 28 which also comprises the step of administering an immune checkpoint inhibitor to the subject, which immune checkpoint inhibitor inhibits a non-ITIM-mediated pathway.

30. A method according to claim 29, wherein the immune checkpoint inhibitor is or comprises a CTLA4 pathway inhibitor.

31. A method according to claim 30 wherein the CTLA4 pathway inhibitor is a CTLA4 antibody.

32. A method according to claim 28, which comprises the following steps:
(i) isolation of a cell containing sample from a subject;
(ii) transduction or transfection of the cells with a nucleic acid sequence according to claim 23; a nucleic acid construct according to claim 24; or a vector according to claim 25; and
(iii) administration the cells from (ii) to the subject.

33. (canceled)

34. A method according to claim 28, wherein the disease is cancer.

35. A method for making a cell according to claim 20, which comprises the step of introducing a vector according to claim 25 into the cell.

36. A method according to claim 35, wherein the cell is from a sample isolated from a subject.


SEQUENCE LISTING (24 sequences)

SEQ ID NO: 1 - protein, 595 aa, Homo sapiens
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 2 - protein, 214 aa, Homo sapiens
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYY

SEQ ID NO: 3 - protein, 97 aa, Homo sapiens
WFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPL

SEQ ID NO: 4 - protein, 104 aa, Homo sapiens
WYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPY

SEQ ID NO: 5 - protein, 597 aa, Homo sapiens
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIVDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLVDQTSGDQSPLPPCTPTPPCAEMREDSARVYENVGLMQQQRSFR

SEQ ID NO: 6 - protein, 97 aa, Homo sapiens
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPL

SEQ ID NO: 7 - protein, 105 aa, Homo sapiens
WFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 8 - protein, 211 aa, Homo sapiens
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 9 - protein, 272 aa, Homo sapiens
FWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 10 - protein, 275 aa, Homo sapiens
FWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMA

SEQ ID NO: 11 - protein, 272 aa, Artificial Sequence
  variant phosphatase sequence
FWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHSSAGIGRTGTIIVIDMLMENISTKGLDCDIDIQKTIQMVRAQRSGMVQTEAQYKFIYVAIAQF

SEQ ID NO: 12 - protein, 275 aa, Artificial Sequence
  variant phosphatase sequence
FWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHSSAGIGRTGTFIVIDILIDIIREKGVDCDIDVPKTIQMVRSQRSGMVQTEAQYRFIYMA

SEQ ID NO: 13 - protein, 72 aa, Artificial Sequence
  amino terminal sequence of lck
MGCGCSSHPEDDWMENIDVCENCHYPIVPLDGKGTLLIRNGSEVRDPLVTYEGSNPPASPLQDNLVIALHSY

SEQ ID NO: 14 - protein, 20 aa, Foot-and-mouth disease virus
RAEGRGSLLTCGDVEENPGP

SEQ ID NO: 15 - protein, 20 aa, Foot-and-mouth disease virus
QCTNYALLKLAGDVESNPGP

SEQ ID NO: 16 - protein, 6 aa, Artificial Sequence
  ITIM conserved sequence
  MISC_FEATURE (1)..(1): Xaa may be Ser, Ile, Val or Leu
  misc_feature (2)..(2): Xaa can be any naturally occurring amino acid
  misc_feature (4)..(5): Xaa can be any naturally occurring amino acid
  MISC_FEATURE (6)..(6): Xaa may be Ile, Val or Leu
XXYXXX

SEQ ID NO: 17 - protein, 5 aa, Artificial Sequence
  alternative SHP-1 sequence
MLSRG

SEQ ID NO: 18 - protein, 5 aa, Homo sapiens
SLSVR

SEQ ID NO: 19 - protein, 5 aa, Artificial Sequence
  alternative SHP-1 sequence
MLSRG

SEQ ID NO: 20 - protein, 10 aa, Homo sapiens
HKEDVYENLH

SEQ ID NO: 21 - protein, 10 aa, Homo sapiens
EKSKGSLKRK

SEQ ID NO: 22 - protein, 10 aa, Artificial Sequence
  alternative SHP-1 sequence
SLESSAGTVA

SEQ ID NO: 23 - protein, 10 aa, Artificial Sequence
  alternative SHP-1 sequence
CTLRTRGRRK

SEQ ID NO: 24 - protein, 8 aa, Artificial Sequence
  myristoylation consensus site
  misc_feature (2)..(4): Xaa can be any naturally occurring amino acid
  misc_feature (6)..(8): Xaa can be any naturally occurring amino acid
GXXXSXXX